(19) United States
(12) Patent Application Publication
(10) Pub. No.: US 2024/0131462 A1
(43) Pub. Date: Apr. 25, 2024

(54) AIR-CLEANING FILTER HAVING DUST COLLECTING AND DEODORIZING FUNCTIONS AND PREPARATION METHOD THEREFOR

(71) Applicant: COWAY Co., Ltd., Gongju-si (KR)

(72) Inventors: Hyun Jun YUN, Seoul (KR); Byong Hyoek LEE, Seoul (KR); Jong Cheol KIM, Seoul (KR)

(73) Assignee: COWAY Co., Ltd., Gongju-si (KR)

(21) Appl. No.: 18/546,822

(22) PCT Filed: Jan. 27, 2022
(86) PCT No.: PCT/KR2022/001464

§ 371 Date: Aug. 16, 2023

(30) Foreign Application Priority Data
Feb. 26, 2021 (KR) 10-2021-0026907

(51) Int. Cl. B01D 46/00; A61L 9/014; B01D 39/16; B01D 46/52; B01D 53/58; B01D 53/72; B01D 53/82; B01D 53/88; B32B 3/28; B32B 5/02; B32B 5/26; B32B 7/12; B32B 37/12; B32B 37/18

(52) U.S. Cl. CPC B01D 46/0036; A61L 9/014; B01D 39/1623; B01D 46/0038; B01D 46/521; B01D 53/58; B01D 53/72; B01D 53/82; B01D 53/885; B32B 3/28; B32B 5/022; B32B 5/269; B32B 5/271; B32B 7/12; B32B 37/1207; B32B 37/185; A61L 2209/14; B01D 2239/0407; B01D 2239/045; B01D 2239/0478; B01D 2239/0618; B01D 2239/0622; B01D 2239/0627; B01D 2239/0668; B01D 2239/10; B01D 2239/1291; B01D 2251/2067; B01D 2251/512; B01D 2257/406; B01D 2257/7027; B01D 2257/708; B01D 2257/90; B01D 2258/06; B01D 2275/10; B32B 2037/1215; B32B 2250/20; B32B 2255/02; B32B 2255/20; B32B 2255/24; B32B 2262/0253; B32B 2262/0276; B32B 2305/20; B32B 2305/28; B32B 2307/718; B32B 2323/10; B32B 2367/00

(57) ABSTRACT

An air-cleaning filter includes a non-woven fabric for dust collection and a non-woven fabric for deodorization having activated carbon particles combined therewith. By adjusting the particle diameter and content of the activated carbon particles and laminating with a non-woven fabric coated with an absorber for other gases, a dust collecting function and a deodorizing function can be imparted at the same time while also creating a structure with a low differential pressure.

### TECHNICAL FIELD

The present invention relates to an air purifying filter having dust collection and deodorization functions and a process for manufacturing the same. More specifically, the present invention relates to an air purifying filter having a combination of dust collection and deodorization functions, a process for manufacturing the same, an air purifying filter for deodorization adopted therein, and an air purifier comprising the same.

### BACKGROUND ART

As air pollution problems such as fine dust and yellow dust have emerged in recent years, air purification that filters indoor air has become essential. An air purifying device such as an air purifier is capable of supplying fresh air by filtering out contaminated dust or substances harmful to the human body in the air by using various filter systems.

Various dust collecting filters adopting a filter medium for removing particulate pollutants contained in a gas are used in such filter systems. In general, a filter medium provided with a polytetrafluoroethylene membrane or a filter medium adopting a melt-blown nonwoven fabric is used in such dust collecting filters.

In addition, a filter system may be provided with a deodorizing filter to remove odor components such as formaldehyde, toluene, and ammonia in the air. For example, a filter medium for adsorbing odor components in which activated carbon is coated on a thermal-bonded or spun-bonded nonwoven fabric has been developed in order to remove formaldehyde.

Further, with the recent trend of air purifiers becoming more complex, air purifying filters that combine dust collection and deodorization functions and other additional functions are being developed.

### DISCLOSURE OF INVENTION

### Technical Problem

Conventional composite air cleaning filters implement dust collection and deodorization functions by simply combining a deodorizing filter layer using pellet-type activated carbon particles and a dust collecting filter layer using a melt-blown nonwoven fabric. However, there is a problem in such a conventional filter in that the differential pressure is increased, which ultimately lowers the cleaning efficiency of the filter.

An object of the present invention is to provide an air purifying filter having excellent dust collection and deodorization performance and implementing low differential pressure, a process for manufacturing the same, and an air purifier comprising the same.

### Solution to Problem

The air purifying filter according to the present invention comprises a pleated filter medium, wherein the filter medium comprises a dust collecting filter layer comprising a first nonwoven fabric; a deodorizing filter layer comprising a second nonwoven fabric and activated carbon particles having a particle size smaller than 150 mesh bound to the second nonwoven fabric in an amount of 10 g/m² to 60 g/m²; and a functional filter layer comprising a third nonwoven fabric and at least one gas adsorbent, wherein the dust collecting filter layer, the deodorizing filter layer, and the functional filter layer are physically or chemically laminated.

The process for manufacturing an air purifying filter according to the present invention comprises preparing a dust collecting filter layer comprising a first nonwoven fabric; binding activated carbon particles having a particle size smaller than 150 mesh in an amount of 10 g/m² to 60 g/m² to a second nonwoven fabric to prepare a deodorizing filter layer; preparing a functional filter layer comprising a third nonwoven fabric and at least one gas adsorbent; physically or chemically laminating the dust collecting filter layer, the deodorizing filter layer, and the functional filter layer to prepare a filter medium; and pleating the filter medium.

In addition, the air purifying filter according to the present invention comprises a nonwoven fabric and activated carbon particles bound to the nonwoven fabric by a binder, wherein the nonwoven fabric is a spun-bonded or thermal-bonded nonwoven fabric having a basis weight of 30 g/m² to 80 g/m², the activated carbon particles have a particle diameter smaller than 150 mesh and are employed in an amount of 10 g/m² to 60 g/m², and the binder is employed in an amount of 20 to 50 parts by weight relative to 100 parts by weight of the activated carbon particles.

In addition, the present invention provides an air purifier comprising the air purifying filter.

### Advantageous Effects of Invention

The air purifying filter according to the present invention comprises a nonwoven fabric for dust collection and a nonwoven fabric in which activated carbon particles are bound for deodorization, while the particle size and content of the activated carbon particles are adjusted, and they are laminated with a nonwoven fabric containing a gas adsorbent; thus, it is possible to impart dust collection and deodorization functions at the same time and to implement a low differential pressure structure.

Specifically, the air purifying filter according to the present invention is provided with a deodorizing filter in which activated carbon particles having a smaller particle size than conventional ones are bound to a nonwoven fabric with a binder, whereby it can adsorb odor components such as toluene at a low differential pressure.

In addition, the air purifying filter according to the present invention may further comprise one or more functional filter layers or metal catalyst layers depending on the components in the air to be removed, and the layers may be laminated in various orders to exhibit composite performance.

### BRIEF DESCRIPTION OF DRAWING

FIG. 1 shows a perspective view of an air purifying filter according to an embodiment of the present invention.

FIG. 2 is a cross-sectional view taken along line A-A′ in FIG. 1 and an enlarged view thereof.

FIG. 3 is a cross-sectional view taken along line B-B′ in FIG. 2.

FIGS. 4 to 6 show cross-sectional views of filter media according to other embodiments.

FIG. 7 shows the results of the gas removal test of the air purifying filter in Test Example 1.

FIG. 8 shows the results of the deodorization endurance test of the air purifying filter in Test Example 2.

FIG. 9 shows the results of the dust collection efficiency test of the air purifying filter in Test Example 3.

FIG. 10 shows the reduction in the concentration of contaminants by the air purifying filter in Test Example 4.

FIG. 11 shows the results of the differential pressure measurement of the air purifying filter in Test Example 5.

### BEST MODE FOR CARRYING OUT THE INVENTION

Hereinafter, embodiments of the present invention will be described in detail with reference to the drawings.

When it is determined that a detailed description of a related known constitution or function may obscure the gist of the present invention in describing the present invention, a detailed description thereof will be omitted. In addition, for the sake of description, the sizes of individual elements in the appended drawings may be exaggeratedly depicted or omitted, and they may differ from the actual sizes.

In the present specification, in the case where an element is mentioned to be formed, connected, or combined on or under another element, it means all of the cases where one element is directly, or indirectly through another element, formed, connected, or combined with another element. In addition, it should be understood that the criterion for the terms on and under of each component may vary depending on the direction in which the object is observed.

In this specification, terms referring to the respective components are used to distinguish them from each other and are not intended to limit the scope of the present invention. In addition, in the present specification, a singular expression is interpreted to cover a plural number as well unless otherwise specified in the context.

In the present specification, the term “comprising” is intended to specify a particular characteristic, region, integer, step, operation, element, and/or component. It does not exclude the presence or addition of any other characteristic, region, integer, step, operation, element and/or component, unless specifically stated to the contrary.

Air Purifying Filter

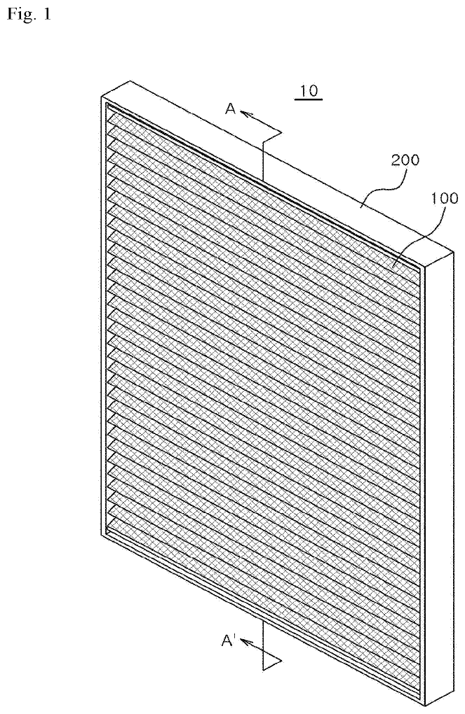

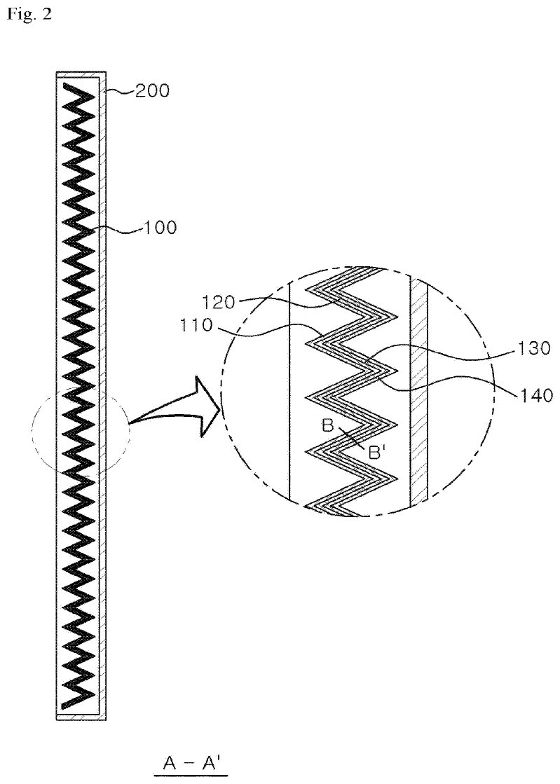

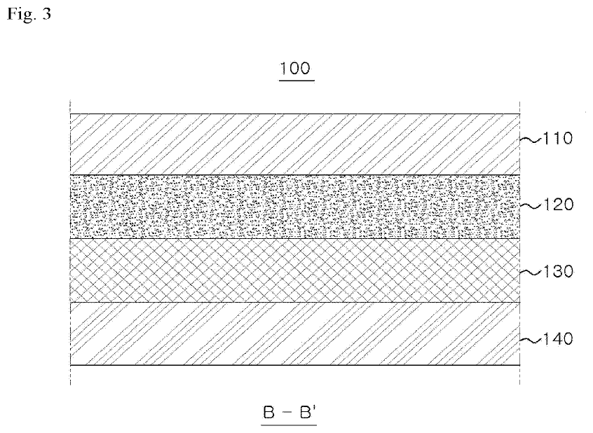

FIG. 1 shows a perspective view of an air purifying filter according to an embodiment of the present invention. FIG. 2 is a cross-sectional view taken along line A-A′ in FIG. 1 and an enlarged view thereof. FIG. 3 is a cross-sectional view taken along line B-B′ in FIG. 2.

Referring to FIGS. 1 to 3, the air purifying filter (10) according to an embodiment of the present invention comprises a pleated filter medium (100), wherein the filter medium (100) comprises a dust collecting filter layer (130) comprising a first nonwoven fabric; a deodorizing filter layer (120) comprising a second nonwoven fabric and activated carbon particles having a particle size smaller than 150 mesh bound to the second nonwoven fabric in an amount of 10 g/m² to 60 g/m²; and a functional filter layer (110) comprising a third nonwoven fabric and at least one gas adsorbent.

The air purifying filter according to an embodiment of the present invention comprises a nonwoven fabric for dust collection and a nonwoven fabric in which activated carbon particles are bound for deodorization, while the particle size and content of the activated carbon particles are adjusted, and they are laminated with a nonwoven fabric coated with a gas adsorbent; thus, it is possible to impart dust collection and deodorization functions at the same time and to implement a low differential pressure configuration. Specifically, the air purifying filter is provided with a deodorizing filter layer in which activated carbon particles having a smaller particle size than conventional ones are bound to a nonwoven fabric with a binder, whereby it can adsorb odor components such as toluene at a low differential pressure.

In addition, the air purifying filter according to an embodiment of the present invention may further comprise one or more functional filter layers or metal catalyst layers depending on the components in the air to be removed, which may be laminated in various orders to exhibit composite performance. Specifically, the air purifying filter (10) may further comprise a metal catalyst layer (140).

Hereinafter, each component layer will be described in detail.

Dust Collecting Filter Layer

The dust collecting filter layer comprises a first nonwoven fabric.

For example, the first nonwoven fabric may be a melt-blown nonwoven fabric.

The first nonwoven fabric may have a basis weight of, for example, 20 g/m² to 40 g/m², specifically, 20 g/m² to 35 g/m², more specifically, 25 g/m² to 35 g/m². If the basis weight of the first nonwoven fabric is within the above preferred range, it may be more advantageous for a pleating process for manufacturing a filter while achieving a high efficiency particulate air (HEPA) grade and securing endurance.

Specifically, the first nonwoven fabric may be a melt-blown nonwoven fabric having a basis weight of 20 g/m² to 35 g/m².

The material of the first nonwoven fabric preferably has a melt index in a specific range. The melt index may be, for example, 800 g/10 minutes to 1,500 g/10 minutes at 265° C., specifically, 900 g/10 minutes to 1,200 g/10 minutes at 265° C., more specifically, 950 g/10 minutes to 1,200 g/10 minutes. Within the above melt index range, processability and productivity at low temperatures can be further enhanced.

As an example, the material of the first nonwoven fabric may be polypropylene. Specifically, it may be polypropylene having a melt index of 800 g/10 minutes to 1,500 g/10 minutes at 265° C.

Meanwhile, the dust collecting filter layer contains ultrafine fibers having a high collection efficiency even when the size of fine dust particles is several microns or less. Thus, particles smaller than the pores can also be removed by using electrostatics, thereby increasing the removal efficiency of pollutants, and the high porosity makes it possible to operate with a low differential pressure (pressure loss).

Deodorizing Filter Layer

The deodorizing filter layer comprises a second nonwoven fabric and activated carbon particles having a particle size smaller than 150 mesh bound to the second nonwoven fabric in an amount of 10 g/m² to 60 g/m².

The material of the second nonwoven fabric may be a polymer resin having excellent thermal resistance and may comprise, for example, polyethylene terephthalate (PET). If the second nonwoven fabric comprises PET having excellent thermal resistance, it is possible to minimize the deterioration in the performance after lamination by heating and to further enhance the adhesion to adjacent layers.

The second nonwoven fabric may have a basis weight of, for example, 20 g/m² to 90 g/m², specifically, 30 g/m² to 80 g/m², more specifically, 40 g/m² to 70 g/m². Within the above preferred range, it is possible to further enhance the sustainability, processability, pleatability, high-temperature processability, and adhesive strength of the filter medium.

Specifically, the second nonwoven fabric may be a spun-bonded or thermal-bonded nonwoven fabric having a basis weight of 30 g/m² to 80 g/m².

The sum of the basis weight of the first nonwoven fabric and the basis weight of the second nonwoven fabric may be 35 g/m² to 85 g/m². For example, the sum of the basis weight of the first nonwoven fabric and the basis weight of the second nonwoven fabric may be 40 g/m² to 80 g/m² or 50 g/m² to 80 g/m². Within the above preferred range, it may be advantageous for securing pleatability and processability with excellent endurance.

The activated carbon particles are obtained from coal or coconut shells and can function to adsorb and remove tobacco smoke, volatile organic compounds, and other odorous substances.

As the particle diameter of the activated carbon particles is smaller than that of 150 mesh, it is advantageous for implementing excellent deodorization performance and low differential pressure. For example, the particle diameter of the activated carbon particles may be 150 mesh or less, 100 mesh or less, or 50 mesh or less, and may be 300 mesh or more, 250 mesh or more, or 200 mesh or more. Specifically, the particle diameter of the activated carbon particles may be 50 mesh to 300 mesh, 100 mesh to 300 mesh, or 150 mesh to 250 mesh. The particle diameter of the activated carbon particles exemplified above may be an average particle diameter.

The surface area of the activated carbon particles is not particularly limited as long as it has physical or chemical properties suitable for the purpose of removing odors. When the applicability to fields such as air purifiers is taken into consideration, they may have a BET surface area of about 1,000 m²/g or more, preferably, about 1,000 to 1,200 m²/g.

The activated carbon particles may be bound to the second nonwoven fabric by a binder. For example, an acrylic type or a polyurethane type may be used as the binder.

In addition, the amount of the binder used may be 10 parts by weight or more, 20 parts by weight or more, or 30 parts by weight or more, and may be 60 parts by weight or less, 50 parts by weight or less, or 40 parts by weight or less, relative to 100 parts by weight of the activated carbon particles. Preferably, it may preferably be 20 to 50 parts by weight from the viewpoint of fixing the activated carbon particles without increasing the differential pressure.

The deodorizing filter layer can implement a low differential pressure configuration while exhibiting superior deodorizing performance as compared with conventional deodorizing filters. Thus, the deodorizing filter layer itself may be provided as an air purifying filter distinguished from the conventional ones.

Specifically, the deodorizing filter layer comprises a nonwoven fabric and activated carbon particles bound to the nonwoven fabric by a binder, wherein the nonwoven fabric is a spun-bonded or thermal-bonded nonwoven fabric having a basis weight of 30 g/m² to 80 g/m², the activated carbon particles have a particle diameter smaller than 150 mesh and are employed in an amount of 10 g/m² to 60 g/m², and the binder is employed in an amount of 20 to 50 parts by weight relative to 100 parts by weight of the activated carbon particles.

Functional Filter Layer

The functional filter layer comprises a third nonwoven fabric and at least one gas adsorbent.

The material of the third nonwoven fabric may be a polymer resin having excellent thermal resistance and may comprise, for example, polyethylene terephthalate (PET). If the third nonwoven fabric comprises PET having excellent thermal resistance, it is possible to minimize the deterioration in the performance after lamination by heating and to further enhance the adhesion to adjacent layers.

The third nonwoven fabric may have a basis weight of, for example, 20 g/m² to 90 g/m², specifically, 30 g/m² to 80 g/m², more specifically, 40 g/m² to 70 g/m². Within the above preferred range, it is possible to further enhance the sustainability, processability, pleatability, high-temperature processability, and adhesive strength of the filter medium. Specifically, the third nonwoven fabric may be a spun-bonded or thermal-bonded nonwoven fabric having a basis weight of 30 g/m² to 80 g/m².

The functional filter layer may remove at least one type of gas depending on the gas adsorbent contained therein. For example, the functional filter layer may remove at least one gas selected from the group consisting of ammonia, aldehydes, acetic acid, toluene, and terpenes. In addition, the gas adsorbent may comprise at least one selected from the group consisting of organic acids, inorganic acids, urea, silica, zeolite, and metal catalysts.

As an example, the functional filter layer may comprise an organic acid or an inorganic acid to remove basic gas such as ammonia gas. As another example, the functional filter layer may comprise urea such as ethylene urea to remove aldehydes. As another example, the functional filter layer may comprise urea together with an organic acid or an inorganic acid to simultaneously remove basic gas and aldehydes.

The content of the gas adsorbent in the functional filter layer may be 10% by weight or more, 2% by weight or more, or 5% by weight or more, and may be 50% by weight or less, 30% by weight or less, or 20% by weight or less.

Specifically, the functional filter layer may comprise at least one selected from 2% by weight to 20% by weight of phosphoric acid for removing ammonia and 5% by weight to 30% by weight of ethylene urea for removing aldehydes.

Metal Catalyst Layer

The filter medium may further comprise a metal catalyst layer, and the metal catalyst layer may comprise a fourth nonwoven fabric and a metal catalyst.

The material of the fourth nonwoven fabric may be a polymer resin having excellent thermal resistance and may comprise, for example, polyethylene terephthalate (PET). If the fourth nonwoven fabric comprises PET having excellent thermal resistance, it is possible to minimize the deterioration in the performance after lamination by heating and to further enhance the adhesion to adjacent layers.

The fourth nonwoven fabric may have a basis weight of, for example, 20 g/m² to 90 g/m², specifically, 30 g/m² to 80 g/m², more specifically, 40 g/m² to 70 g/m². Within the above preferred range, it is possible to further enhance the sustainability, processability, pleatability, high-temperature processability, and adhesive strength of the filter medium.

The metal catalyst layer can decompose and remove gases that are difficult to adsorb and remove through the reaction of the metal catalyst.

For example, the gas to be removed by the metal catalyst layer may be at least one selected from the group consisting of formaldehyde, acetaldehyde, and acetic acid.

The metal catalyst may be at least one selected from the group consisting of platinum, copper, and manganese.

The amount of the metal catalyst in the metal catalyst layer may be, for example, 0.1% by weight to 2% by weight.

As an example, the metal catalyst layer may comprise 0.1% by weight to 2% by weight of a platinum catalyst for removing formaldehyde gas.

As another example, the metal catalyst layer may comprise 0.1% by weight to 2% by weight of a manganese catalyst for removing acetaldehyde gas.

Laminated Structure

The filter medium of the air purifying filter according to the present invention may have various laminated configurations in which the above-described layers are physically or chemically laminated.

Preferably, the deodorizing filter layer and the dust collecting filter layer may be laminated adjacent to each other, and the functional filter layer may be laminated on the deodorizing filter layer or the dust collecting filter layer. If the deodorizing filter layer and the dust collecting filter layer are laminated adjacent to each other as described above, even if the activated carbon particles are detached from the deodorizing filter layer due to the inflow of air, they can be preserved by the dust collecting filter layer. As shown in FIG. 3, the filter medium (100) according to an embodiment may be one in which a functional filter layer (110), a deodorizing filter layer (120), a dust collecting filter layer (130), and a metal catalyst layer (140) are sequentially laminated. If the functional filter layer is laminated on the deodorizing filter layer as described above, it may perform the protective function of the deodorizing filter layer, whereby the lifespan of the deodorizing filter layer can be further enhanced.

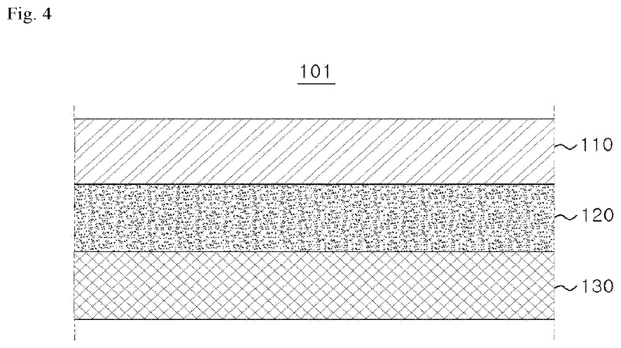

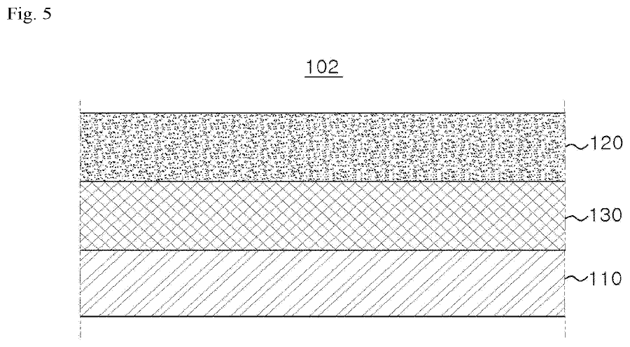

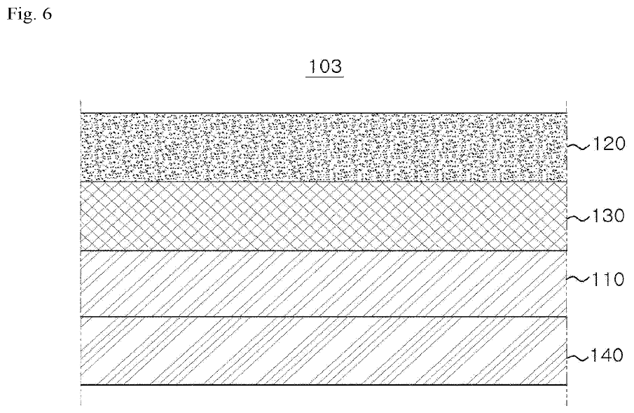

FIGS. 4 to 6 show cross-sectional views of filter media of air purifying filters according to other embodiments. According to FIG. 4, the filter medium (101) may be one in which a functional filter layer (110), a deodorizing filter layer (120), and a dust collecting filter layer (130) are sequentially laminated. According to FIG. 5, the filter medium (102) may be one in which a deodorizing filter layer (120), a dust collecting filter layer (130), and a functional filter layer (110) are sequentially laminated. According to FIG. 6, the filter medium (103) may be one in which a deodorizing filter layer (120), a dust collecting filter layer (130), a functional filter layer (110), and a metal catalyst layer (140) are sequentially laminated.

In addition, the filter medium may comprise two or more functional filter layers. For example, an additional functional filter layer may be laminated on the surface of the dust collecting filter layer (130) in the filter medium (101) of FIG. 4. A composite performance of removing two or more types of gases can be achieved by coating different gas adsorbents on the two or more functional filter layers.

In addition, the air purifying filter is adopted in an air purifier, in which it may be configured such that air flows into the deodorizing filter layer and flows out of the dust collecting filter layer. Therefore, even if the activated carbon particles bound to the deodorizing filter layer are detached from the second nonwoven fabric by the flow of air, they may be captured in the dust collecting filter layer.

Housing

The air purifying filter according to the present invention may further comprise a housing of the filter medium. Referring to FIGS. 1 and 2, the air purifying filter may comprise a housing (200) and a pleated filter medium (100) disposed inside the housing.

The housing may serve as a frame for supporting the filter medium. It may be assembled or molded such that the filter medium may be properly disposed and mounted. The shape or structure of the housing may be arbitrarily determined according to the purpose of use or environment.

The material of the housing may be a material of a conventional housing used for an air purifying filter. Specifically, at least one selected from the group consisting of an acrylonitrile-butadiene-styrene copolymer (ABS), polypropylene (PP), paper, nonwoven fabrics, polycarbonate (PC), and elastomer resins may be used as a material of the housing. More specifically, ABS or PP may be used as the material of the housing, and ABS may be preferably used in consideration of the fact that dimensional accuracy may be readily secured and deformation during use may be suppressed. In addition, polyethylene terephthalate (PET) and ABS have high adhesiveness to each other. Thus, when PET is used as a material for the outer layer of the filter medium, and ABS is used as a material for the housing, the prevention of delamination of the filter medium and the housing may be enhanced.

The filter medium may be disposed inside the housing once it has been molded.

Characteristics of the Air Purifying Filter

The air purifying filter according to the present invention can implement a low differential pressure while maintaining high removal efficiency of pollutants in the air, whereby it can have excellent lifespan characteristics.

In general, the lifespan of a filter and the differential pressure of the filter are highly affected by each other. The differential pressure of a filter means the difference in pressure between the upstream and the downstream of the filter medium. When a fluid containing contaminated particles passes through a filter, the particles are collected in the pores of the filter to clog the pores. When the pores are clogged, it gradually increases the pressure. That is, the differential pressure of a filter is gradually increased as time passes or as particles are collected in the filter. Thus, the differential pressure of a filter is a main factor that determines the replacement timing of the filter and may be a measure for determining the lifespan of the filter.

If the differential pressure of a filter is too low, the performance of pollutant removal may be deteriorated. On the other hand, if the differential pressure of a filter is too high, the lifespan of the filter may be shortened, and power consumption may increase. Thus, to have a differential pressure in an appropriate range may be very advantageous for satisfying high performance, low power consumption, and high lifespan characteristics of a filter at the same time.

For example, the differential pressure of the air purifying filter may be 40 Pa or more, 50 Pa or more, 60 Pa or more, or 70 Pa or more, and may be 120 Pa or less, 110 Pa or less, 100 Pa or less, or 90 Pa or less, under the condition of a dust supply of 50 g at a flow rate of 1 m/s. As a specific example, the differential pressure of the air purifying filter may be 60 Pa to 100 Pa under the condition of a dust supply of 50 g at a flow rate of 1 m/s.

The air purifying filter may have a removal rate of 90% for all of formaldehyde, toluene, and ammonia when a gas removal test is performed for 30 minutes under the conditions of a temperature of 25° C., a humidity of 50%, a chamber size of 8 m³, and an initial concentration of 10 ppm of a target gas.

Specifically, the air purifying filter may have a removal rate of 93% or more for formaldehyde, a removal rate of 95% or more for toluene, and a removal rate of 97% or more for ammonia, when a gas removal test is performed for 30 minutes under the above conditions.

In addition, the air purifying filter can maintain a removal rate of 80% or more for all of formaldehyde, toluene, and ammonia even when the number of cigarettes is increased to 100 in the deodorization endurance test under the conditions of JEM1467:2009. In particular, the air purifying filter can maintain a removal rate of 95% or more for ammonia even if the number of cigarettes is increased to 200; thus, the deodorization endurance may be very excellent.

In addition, the air purifying filter may have a dust collection efficiency of 99% or more, 99.5% or more, 99.6% or more, 99.7% or more, or 99.8% or more, under the conditions of 42 CFR part 84 using di-ethyl-hexyl-sebacat (DEHS) particles or NaCl particles.

Process for Manufacturing the Air Purifying Filter

The process for manufacturing an air purifying filter according to the present invention comprises preparing a dust collecting filter layer comprising a first nonwoven fabric; binding activated carbon particles having a particle size smaller than 150 mesh in an amount of 10 g/m² to 60 g/m² to a second nonwoven fabric to prepare a deodorizing filter layer; preparing a functional filter layer comprising a third nonwoven fabric and at least one gas adsorbent; physically or chemically laminating the dust collecting filter layer, the deodorizing filter layer, and the functional filter layer to prepare a filter medium; and pleating the filter medium.

The first nonwoven fabric of the dust collecting filter layer may be prepared using the material exemplified above. For example, it may be prepared to have a basis weight of 20 g/m² to 35 g/m² by a melt-blown method using polypropylene.

The deodorizing filter layer may be prepared by binding activated carbon particles to a second nonwoven fabric using a binder. Specifically, it may be prepared by mixing activated carbon particles with a binder and a solvent to prepare a dispersion, which may be coated onto the second nonwoven fabric and dried.

In such an event, the binder may be employed in an amount of 20 to 50 parts by weight relative to 100 parts by weight of the activated carbon particles.

In addition, distilled water, purified water, or the like may be used as the solvent in an amount of 200 to 500 parts by weight relative to 100 parts by weight of the activated carbon particles.

The coating may be carried out by spraying the dispersion prepared above onto the second nonwoven fabric or immersing the second nonwoven fabric in the dispersion. Specifically, the coating may be carried out by immersing the second nonwoven fabric in the dispersion for 3 to 10 seconds.

In addition, the drying may be carried out at a temperature of 50° C. to 70° C. for 5 minutes to 10 minutes.

The functional filter layer may be prepared by coating one or more types of gas adsorbents on a third nonwoven fabric, in which it may be coated with the gas adsorbents and contents exemplified above.

The second nonwoven fabric and the third nonwoven fabric may each be prepared using the material exemplified above. For example, it may be prepared to have a basis weight of 30 g/m² to 80 g/m² using polyethylene terephthalate in a spun-bonding or thermal-bonding method, respectively.

The lamination is carried out in a physical or chemical manner. For example, the lamination may be carried out through physical lamination by pressure or chemical lamination using an adhesive or hot melt.

Specifically, if a hot melt is used for the lamination, it may be carried out at a temperature of 130° C. to 170° C. More specifically, the lamination using a hot melt may be carried out at a temperature of 130° C. to 170° C., 140° C. to 160° C., or 150° C. to 170° C.

When a hot melt is used, the amount of the hot melt may be 1 g/m² to 10 g/m², 2 g/m² to 10 g/m², or 3 g/m² to 8 g/m². If the hot melt adhesive is used in the above range, adhesion may be enhanced. For example, acrylic, polyolefin, polyester, polyamide, polyurethane, or the like may be used as the hot melt.

Thereafter, the filter medium is pleated. If the filter medium is pleated, the filtration area is broadened to reduce the pressure loss, and its structure is solid to enhance the endurance and lifespan characteristics of the filter.

Referring to FIG. 2, the filter medium (100) may be bent into a pleated shape using a rotary pleating machine or the like. For example, the pleated shape may be a structure in which pleats are formed by bending. In addition, the shape of the pleats may vary, such as a zigzag type angular pleat or rounded pleat. The shape and size of the pleats are not particularly limited.

In addition, the height of the peaks of the pleats may be 10 mm to 60 mm. Here, the height of the peaks may refer to the amplitude of the pleats, that is, the distance between the peak and the valley. In addition, the distance between the peaks may be 2 mm to 8 mm.

Air Purifier

The present invention provides an air purifier comprising the air purifying filter described above.

As an example, the air purifier comprises an inlet for introducing polluted air; an outlet for discharging purified air; and a filter unit disposed between the inlet and the outlet, wherein the filter unit comprises the air purifying filter described above.

Specifically, the air purifier may be provided with an inlet in the front for introducing indoor air, an outlet formed at the upper part for discharging purified air, and a filter unit comprising the air purifying filter inside thereof.

In addition, the air purifier may be provided with a blower fan that introduces indoor air by a rotational force and discharges purified air to the room. The blower fan introduces air through the front inlet and discharges it through the upper outlet. The outlet is provided with an outlet grill having a dense grid shape. As a result, it is possible to prevent the user's body from being injured by the rotating blower fan.

The filter unit may further comprise an additional filter in addition to the air purifying filter. For example, it may further comprise a pre-filter constructed using an antibacterial material for removing relatively large dust, mold, hair, pet hair, and the like and/or a dehumidifying filter having a plurality of pores to remove moisture in the air.

### MODE FOR THE INVENTION

### Example

Hereinafter, although embodiments of the present invention are described, the scope of the present invention to be implemented is not limited thereto.

### Example 1: Manufacturing of an Air Purifying Filter (Composite Filter)

(1) Preparation of a Dust Collecting Filter Layer

A first nonwoven fabric having a basis weight of about 27 g/m² was prepared using a polypropylene (PP) resin by a melt-blown method.

(2) Preparation of a Deodorizing Filter Layer

A second nonwoven fabric having a basis weight of about 55 g/m² was prepared using a polyethylene terephthalate (PET) resin by a spun-bonding method. A dispersion was prepared by mixing 100 parts by weight of coconut shell activated carbon having an average particle diameter of about 200 mesh, 40 parts by weight of a binder (acrylic polyol resin), and 400 parts by weight of a solvent (purified water). The second nonwoven fabric was immersed in the dispersion for 10 seconds, and it was then taken out and dried at 60° C. for 10 minutes. As a result, a deodorizing filter layer in which the second nonwoven fabric was coated with coconut shell activated carbon in an amount of about 35 g/m² was prepared.

(3) Preparation of a Functional Filter Layer

Two sheets of a third nonwoven fabric having a basis weight of about 55 g/m² were prepared using a polyethylene terephthalate (PET) resin by a spun-bonding method.

A functional filter layer A was prepared by coating phosphoric acid in an amount of about 11% by weight onto one of the third nonwoven fabrics.

A functional filter layer B was prepared by coating ethylene urea (2-imidazolidone) in an amount of about 17% by weight onto the other of the third nonwoven fabrics.

(4) Lamination

The functional filter layer A, deodorizing filter layer, dust collecting filter layer, and functional filter layer B were placed in the order from the bottom with the coated surface facing upward, which were laminated at 150° C. using a polyolefin hot melt to obtain a filter medium.

(5) Pleating

The filter medium was pleated using a rotary pleating machine (DBWP-W700, DoubleWin) to have a peak height of 25 mm and a distance between peaks of about 3.5 mm of the pleats. Upon completion of the pleating processing, the filter medium was insert-molded into an ABS housing using a molding machine (Filter Assay M/C, DoubleWin) to manufacture an air purifying filter.

### Comparative Example 1

A dust collecting filter layer was prepared by repeating the procedure of step (1) of Example 1. In addition, a deodorizing filter layer in which pellet-type activated carbon particles are contained in a net was prepared. The dust collecting filter layer and the deodorizing filter layer were simply combined to manufacture an air purifying filter.

### Example 2

An air purifying filter was prepared by repeating the procedure of Example 1, except that the deodorizing filter layer and the dust collecting filter layer were laminated without preparation of the functional filter layer of step (3).

### Test Example 1: Gas Removal Test

The gas removal test was carried out under the test conditions of a temperature of 25° C., a humidity of 50%, a chamber size of 8 m³, and an initial concentration of a target gas of 10 ppm according to KACA002 132:2018 using the air purifying filter of Example 1.

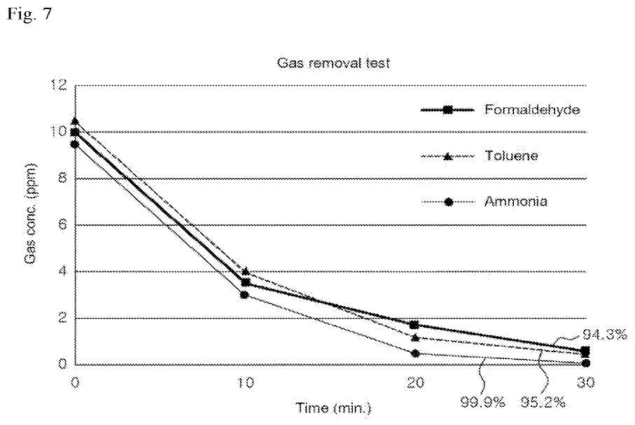

The results are shown in FIG. 7. As shown in FIG. 7, the air purifying filter of Example 1 showed a removal rate of 90% or more after 30 minutes for all of formaldehyde, toluene, and ammonia gas. In particular, it showed the highest removal rate of 99.9% after 30 minutes for ammonia gas.

### Test Example 2: Deodorization Endurance Test

The deodorization endurance test was carried out under the test conditions according to JEM1467:2009 using the air purifying filter of Example 1.

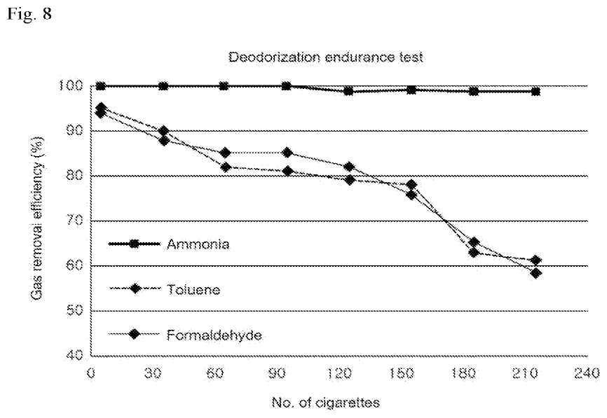

The results are shown in FIG. 8. As shown in FIG. 8, the air purifying filter of Example 1 showed a gradual decrease in the removal efficiency for formaldehyde, toluene, and ammonia gases as the number of cigarettes increased. In particular, the removal efficiency was hardly decreased for ammonia gas, thereby indicating that the deodorization endurance was evaluated as the best.

### Test Example 3: Dust Collection Efficiency Test

The dust collection efficiency test was carried out according to 42 CFR part 84 with NaCl particles using the air purifying filter of Example 1.

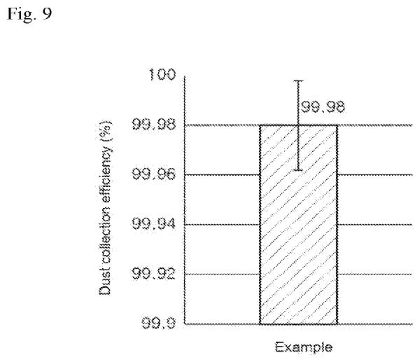

The results are shown in FIG. 9. As shown in FIG. 9, the air purifying filter of Example 1 showed a very high dust collection efficiency of 99.98%.

### Test Example 4: Change in Pollutants Concentration

The test of change in pollutants concentration was carried out according to KACA002 132:2018 using the air purifying filter of Example 1.

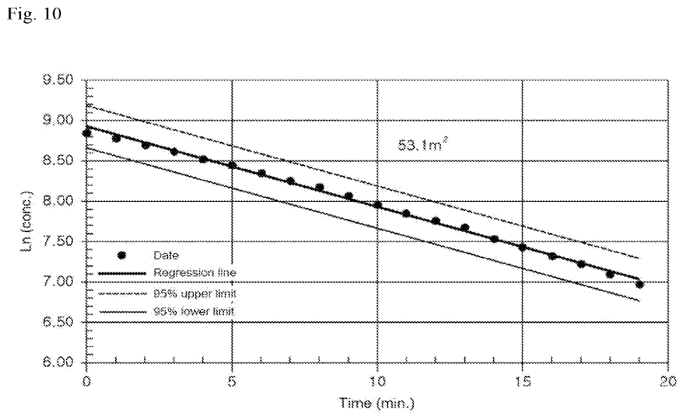

The results are shown in FIG. 10. As shown in FIG. 10, in the air purifying filter of Example 1, the concentration (m³/minute) of KCl linearly decreased exponentially over time.

### Test Example 5: Filter Differential Pressure Test

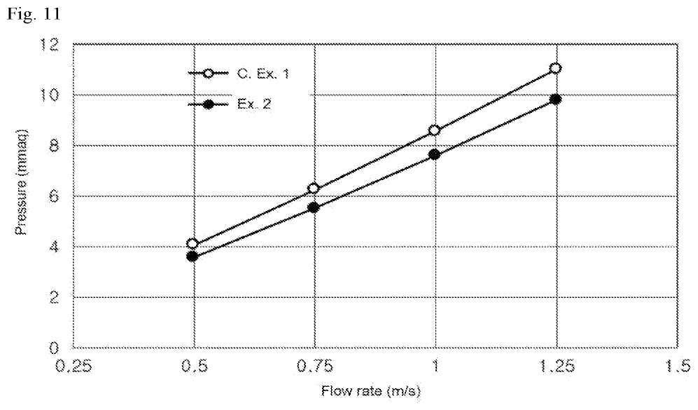

The differential pressure was measured while supplying ISO A2 dust (dust) at a flow rate ranging from 0.5 m/s to 1.25 m/s using a Topas PAF-113-cabin filter test system. The differential pressure of the filter thus measured with respect to the amount of dust supplied is shown in FIG. 11.

As shown in FIG. 11, the air cleaning filter of the present invention in which a deodorizing filter layer thinly coated with fine activated carbon particles and a dust collecting filter layer were laminated had a smaller differential pressure than that of conventional air cleaning filter in which a deodorizing filter layer using pellet-type activated carbon particles and a dust collecting filter layer were simply combined. Specifically, the filter of Example 2 had a differential pressure of about 1 mmaq (about 9.8 Pa) lower than that of the filter of Comparative Example 1 when the dust supply amount was 50 g at a flow rate of 1 m/s.

1. An air purifying filter, which comprises a pleated filter medium, wherein the filter medium comprises:
a dust collecting filter layer comprising a first nonwoven fabric;
a deodorizing filter layer comprising a second nonwoven fabric and activated carbon particles having a particle size smaller than 150 mesh bound to the second nonwoven fabric in an amount of 10 g/m2 to 60 g/m2; and
a functional filter layer comprising a third nonwoven fabric and at least one gas adsorbent,
wherein the dust collecting filter layer, the deodorizing filter layer, and the functional filter layer are physically or chemically laminated.

2. The air purifying filter of claim 1, wherein the first nonwoven fabric is a melt-blown nonwoven fabric having a basis weight of 20 g/m2 to 35 g/m2, and
the second nonwoven fabric and the third nonwoven fabric are a spun-bonded or thermal-bonded nonwoven fabric having a basis weight of 30 g/m2 to 80 g/m2.

3. The air purifying filter of claim 1, wherein the functional filter layer removes at least one gas selected from the group consisting of ammonia, aldehydes, acetic acid, toluene, and terpenes, and
the gas adsorbent comprises at least one selected from the group consisting of organic acids, inorganic acids, urea, silica, zeolite, and metal catalysts.

4. The air purifying filter of claim 1, wherein the functional filter layer comprises at least one selected from phosphoric acid in an amount of 2% by weight to 20% by weight for removing ammonia and ethylene urea in an amount of 5% by weight to 30% by weight for removing aldehydes.

5. The air purifying filter of claim 1, wherein the deodorizing filter layer and the dust collecting filter layer are laminated adjacent to each other, and
the functional filter layer is laminated on the deodorizing filter layer or the dust collecting filter layer.

6. The air purifying filter of claim 5, wherein the air purifying filter is adopted in an air purifier, in which it is configured such that air flows into the deodorizing filter layer and flows out of the dust collecting filter layer.

7. The air purifying filter of claim 1, wherein the filter medium further comprises a metal catalyst layer, and the metal catalyst layer comprises a fourth nonwoven fabric and a metal catalyst.

8. The air purifying filter of claim 7, wherein the functional filter layer, the deodorizing filter layer, the dust collecting filter layer, and the metal catalyst layer are sequentially laminated in the filter medium.

9. The air purifying filter of claim 1, wherein, when a gas removal test is performed for 30 minutes under the conditions of a temperature of 25° C., a humidity of 50%, a chamber size of 8 m3, and an initial concentration of 10 ppm of a target gas, the air purifying filter has a removal rate of 90% for all of formaldehyde, toluene, and ammonia.

10. An air purifying filter, which comprises a nonwoven fabric and activated carbon particles bound to the nonwoven fabric by a binder,
wherein the nonwoven fabric is a spun-bonded or thermal-bonded nonwoven fabric having a basis weight of 30 g/m2 to 80 g/m2,
the activated carbon particles have a particle diameter smaller than 150 mesh and are employed in an amount of 10 g/m2 to 60 g/m2, and
the binder is employed in an amount of 20 to 50 parts by weight relative to 100 parts by weight of the activated carbon particles.

11. The air purifying filter of claim 10, wherein the differential pressure of the air purifying filter is 60 Pa to 100 Pa under the condition of a dust supply of 50 g at a flow rate of 1 m/s.

12. An air purifier, which comprises the air purifying filter of claim 1.

13. A process for manufacturing an air purifying filter, which comprises:
preparing a dust collecting filter layer comprising a first nonwoven fabric;
binding activated carbon particles having a particle size smaller than 150 mesh in an amount of 10 g/m2 to 60 g/m2 to a second nonwoven fabric to prepare a deodorizing filter layer;
preparing a functional filter layer comprising a third nonwoven fabric and at least one gas adsorbent;
physically or chemically laminating the dust collecting filter layer, the deodorizing filter layer, and the functional filter layer to prepare a filter medium; and
pleating the filter medium.

14. An air purifier, which comprises the air purifying filter of claim 2.

15. An air purifier, which comprises the air purifying filter of claim 3.

16. An air purifier, which comprises the air purifying filter of claim 4.

17. An air purifier, which comprises the air purifying filter of claim 5.

18. An air purifier, which comprises the air purifying filter of claim 6.

19. An air purifier, which comprises the air purifying filter of claim 7.

20. An air purifier, which comprises the air purifying filter of claim 8.
